(19) United States
(12) Patent Application Publication
(10) Pub. No.: US 2009/0076394 A1
(43) Pub. Date: Mar. 19, 2009

(54) High-frequency tissue imaging devices and methods

(76) Inventors: William Wong, Milton, MA (US); Alice Chiang, Weston, MA (US); Michael Brodsky, Brookline, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/215,672

(22) Filed: Jun. 27, 2008

Related U.S. Application Data
(60) Provisional application No. 60/937,735, filed on Jun. 29, 2007.

(51) Int. Cl. A61B 8/14; G06K 9/00

(52) U.S. Cl. 600/459; 382/128

(57) ABSTRACT

The present invention relates to systems and methods for signal processing of an ultrasound images. More specifically, a preferred embodiment of an ultrasound imaging system includes a transducer array that provides imaging signals at a first frequency, f₁, and at a second frequency, f₂, into a region of interest. A beamforming system processes image data at a third frequency that is a function of the first frequency, f₁, and at the second frequency, f₂. The processing system processes the ultrasound image using the function of the first frequency, f₁, and the second frequency, f₂. The function of the third frequency is a non-linearly-generated, higher-order, mixing term, f₁+f₂.

### CROSS REFERENCE TO RELATED APPLICATIONS

This application claims priority to U.S. Provisional Application 60/937,735 filed on Jun. 29, 2007, the entire contents of which is incorporated herein by reference.

### BACKGROUND OF THE INVENTION

Ultrasound imaging systems are used for the imaging of internal organs within the human body. These systems typically use a transducer array to deliver energy at a selected frequency and detect the response from a selected region of interest. The collected data is processed to form an image of the region of interest to provide useful diagnostic information.

Due to the inhomogeneous nature of tissue in a body, echo signals received from the reflection of acoustic waves in the tissue are highly non-linear. The non-linear response of the tissue body increases the width of the transmitted-received main beam and the level of the side lobes, which, in turn, significantly decreases the lateral and contrast resolution of ultrasound imaging.

Thus, methods and system are needed for tissue imaging using the propagating wave, to address these defocusing effects.

### SUMMARY OF THE INVENTION

The present invention relates to an ultrasound imaging system. The system includes a transducer array that provides imaging signals at a first frequency, f₁, and at a second frequency, f₂, into a region of interest, such as mammalian tissue; a beamforming system that processes image data at a third frequency that is some function of the first frequency, f₁, and the second frequency, f₂; and a processing system that performs signal processing on the ultrasound imaging data using the image data at the third frequency. More particularly, the processing system performs signal processing of the ultrasound imaging data using a non-linearly-generated, quadratic or higher-order, mixing term, f₁+f₂.

In one aspect of the system, the non-linearly-generated, higher-order, mixing term, f₁+f₂, has a narrower beam width than beam widths associated with either of the imaging signal at frequency, f₁, or the imaging signal at frequency, f₂, and is a quadratic or higher-order term. The processing system includes a bandpass filter that passes a band of frequencies proximate to the bandwidth of the non-linearly-generated, higher-order, mixing term, f₁+f₂, and attenuates frequencies higher and lower than the band of frequencies passed.

Methods of ultrasound imaging and post-imaging processing of ultrasound images are disclosed in which a user can first provide plural imaging input signals at a first frequency, f₁, and at a second frequency, f₂, into a region of interest. Echo signals are then received from the region of interest. In a next step, the methods include filtering and/or processing the detected echo signal at a third frequency that is a function of the first frequency, f₁, and the second frequency, f₂. More particularly, the image data at the third frequency is a non-linearly-generated, quadratic or higher- order, mixing term, f₁+f₂.

In preferred embodiments the methods can be used with ultrasound imaging systems in which the beamformer can be integrated into the transducer scanhead housing, or alternatively, into an interface to a personal computer. The system can be a portable system in which the scanhead interface and computer has a total weight of less than fifteen pounds and preferably under ten pounds, or it can be incorporated into a cart system.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1(a) shows a time domain system response for a linear system;

FIG. 1(b) shows a time domain system response for a non-linear system having quadratic and higher order outputs;

FIG. 2(a) shows a frequency domain response of a non-linear system;

FIG. 2(b) shows a bandpass filter for capturing the high-frequency mixing signal, ƒ₁+ƒ₂, component of the non-linear system;

FIG. 3 shows a block diagram of an ultrasound imaging system in accordance with the present invention;

FIG. 4 shows a phased-array, transmit driver/applicator integrated circuit chip in accordance with the present invention;

FIG. 5 shows a oscilloscope trace of transmitted waveforms of two channels;

FIG. 6 shows a oscilloscope trace of transmitted waveforms containing different frequencies and different number of pulses on a single channel;

FIG. 7 illustrates a first embodiment of an external waveform generator;

FIG. 8 illustrates a second embodiment of an external waveform generation.

FIG. 9 shows a oscilloscope trace of an arbitrary transmitted waveform;

FIG. 10a graphically illustrates the narrowing beam width response of a 64-element phased array transducer at 1.5 MHz, 2.5 MHz, and at 4 MHz;

FIG. 10b illustrates a method of performing an ultrasound imaging sequence in accordance with a preferred embodiments of the invention;

FIG. 11 illustrates a portable ultrasound imaging system in accordance with a preferred embodiment of the invention;

FIG. 12 illustrates the system of claim 11 in a storage position;

FIG. 13 shows an expanded perspective view of the interface module of the system of FIG. 11; and

FIG. 14 is a schematic circuit diagram of the system of FIG. 11 in accordance with a preferred embodiment of the invention.

FIG. 15 illustrates a cart based system utilizing preferred embodiments of the invention.

### DETAILED DESCRIPTION OF THE INVENTION

With reference to the figures, the present invention relates to systems and methods for ultrasound imaging. Notably, the present invention may be implemented using software, hardware or any combination thereof, as would be apparent to those skilled in the art.

Therefore, unless otherwise specified, illustrated embodiments can be understood as providing exemplary features of varying detail of certain embodiments, and therefore, unless otherwise specified, features, components, modules, and/or aspects of the illustrations can be otherwise combined, separated, interchanged, and/or rearranged without departing from the disclosed systems and methods. Additionally, the shapes and sizes of components are also exemplary and unless otherwise specified, can be altered without affecting the disclosed systems or methods. Accordingly, illustrative figures and/or examples used hereinbelow are not meant to limit the scope of the present invention or its embodiments or equivalents.

Tissue High-Frequency Imaging (THI), or Tissue Fusion Imaging (TFI), for ultrasound imaging relates to systems and methods for delivering a plurality of frequencies onto a region of interest and detecting and processing the response of a selected function of the plurality of frequencies, such as the sum of two frequencies, for example.

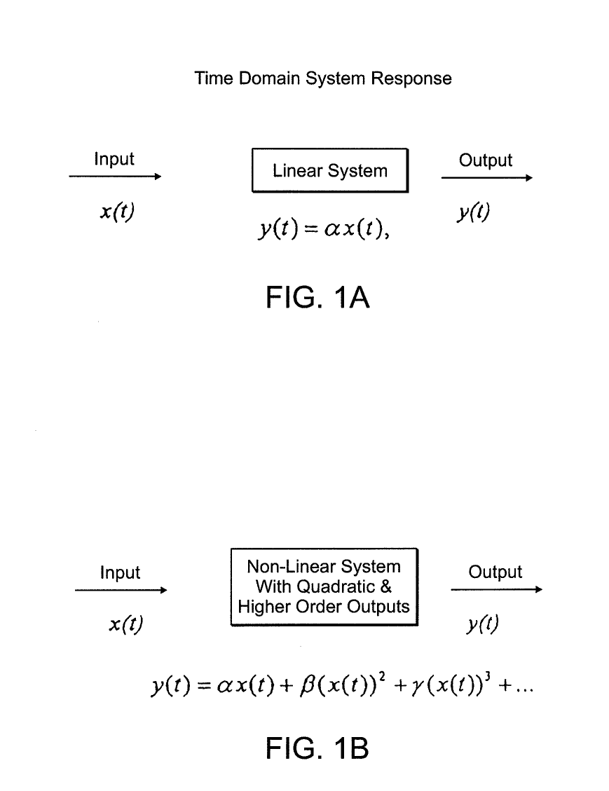

A time domain response of a linear system is shown in FIG. 1(a). Linear systems possess the property of superposition. Hence, given any input, x(t), consisting of several signals, such as x₁(t), x₂(t), and so forth, the system response function, y(t), is merely the superposition of the responses of the system to each of the several input signals. The relationship between the input signal, x(t), and the output (response) signal, y(t), can be expressed as y(t)=αx(t) where a is a constant. Thus, if x(t)=x₁(t)+x₂(t), then y(t)=y₁(t)+y₂(t), where y₁(t)=αx₁(t) and y₂(t)=αx₂(t).

The time domain response of a non-linear system having a quadratic and/or a higher-order non-linear response, is shown in FIG. 1(b). For any input, x(t), the system response function becomes y(t)=αx(t)+β(x(t))²+γ(x(t))³+ . . . , where α,β, γ are all constants. The output of a non-linear system having two simultaneous inputs, x₁(t) and x₂(t), therefore, can be expressed as

y(t)=α(x₁(t)+x₂(t))+β(x₁(t)+x₂(t))²+γ(x₁(t)+x₂(t))³+. [1]

Assuming periodic, sinusoidal input waveforms such as, for example,

x₁(t)=sin2πƒ₁t and x₂(t)=sin2πƒ₂t, [2]

respectively, and substituting Equation [2] into Equation [1], the non-linear system response function can be expressed as:

y(t)=α(sin2πƒ₁t+sin2πƒ₂t)+β(sin2πƒ₁t+sin2πƒ₂t)²+γ(sin2πƒ₁t+sin2πƒ₂t)³+ [3]

The quadratic term can be expanded to become:

(sin2πƒ₁t+sin2πƒ₂t)²=(sin2πƒ₁t)²+(sin2πƒ₂t)²+2sin2πƒ₁tsin2πƒ₂t, [4]

where

2sin2πƒ₁tsin2πƒ₂t=cos2π(ƒ₁−ƒ₂)t−cos2π(ƒ₁+ƒ₂)t. [5]

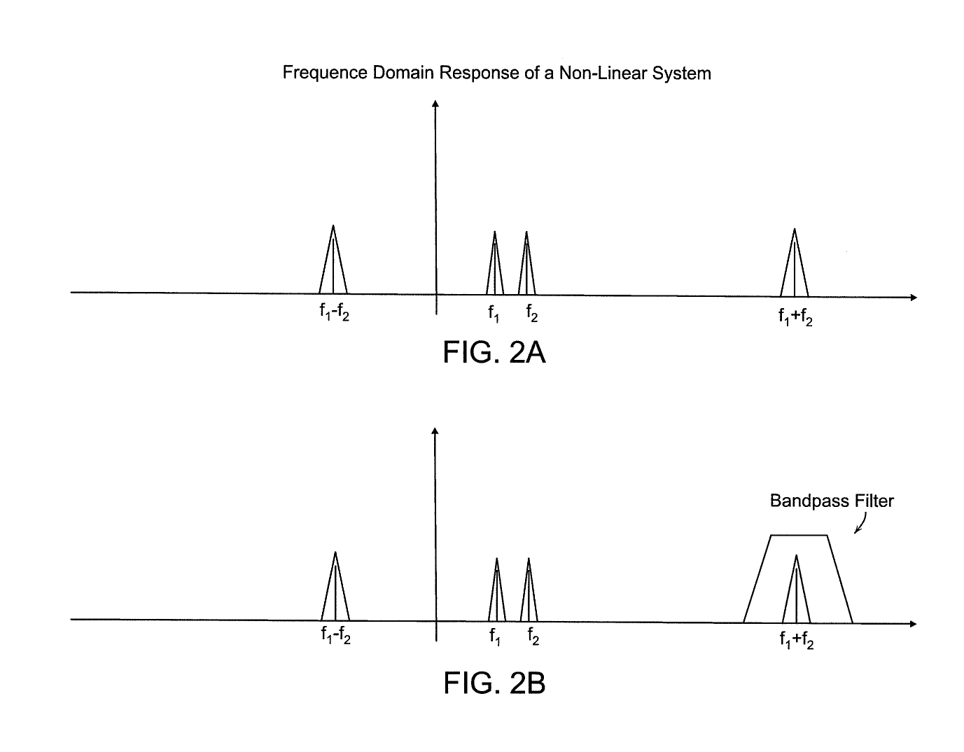

The response of this non-linear system in the frequency domain is shown in FIG. 2(a). As seen in FIG. 2(a), for input waveforms having a spectrum in ƒ₁ and ƒ₂, which is to say having two different frequencies, the frequency response output consists of information in ƒ₁, ƒ₂, ƒ₁−ƒ₂, ƒ₁+ƒ₂, as well as higher order harmonic terms. According to the present invention, a bandpass filter, such as a notch filter, can be used to receive only the high-frequency mixing signal, ƒ₁+ƒ₂. An ultrasound image can then be formed using only the non-linearly-generated, higher-order, quadratic, mixing signal, ƒ₁+ƒ₂.

The received beam pattern of this high-frequency, mixing term has a much narrower beam width than either of the original signals transmitted through the body or tissue. As a result, the high-frequency mixing image based only on the high-frequency, mixing signal ƒ₁+ƒ₂, reduces the beam broadening effect caused by inhomogeneous tissue. Reducing the beam broadening effect yields an image with a better lateral spatial and contrast resolution.

Advantageously, there is no limitation in selecting the input waveforms to the proposed system. For example, if one selects input frequencies such as ƒ₁=1.5 Mhz, ƒ₂=2.5 Mhz, the resulting mixing signal image is formed at ƒ₁+ƒ₂=4 Mhz, or if one selects input frequencies such as ƒ₁=1.6 Mhz, ƒ₂=2.2 Mhz, the resulting mixing signal image is then formed at ƒ₁+ƒ₂=3.8 Mhz.

The bandpass filter can further reduce overall system noise and improve the image quality. Indeed, it is known to those skilled in the art that, the input equivalent noise per unit square root bandwidth of a system with an input equivalent noise resistance, R, is equal to \sqrt{4kTR}, where k is Boltzmann's constant and T is the ambient temperature.

The typical bandwidth, Δf, of a conventional ultrasound receiving system is about 12 Mhz. Hence, assuming that the input equivalent noise resistance, R, of a receiver system is 50 ohms, it follows then the total root-mean-square (rms) noise of the receiver system be equal to

\sqrt{4TR}×(Δf)^{1/2}, or

\sqrt{4kTR}\sqrt{12×10⁶}=3.15 μV.

In contrast, according to a preferred embodiment of the system of the present invention, a bandpass filter with a smaller bandwidth (about 3 Mhz) can be used. As a result, the total rms noise of the received system is reduced to

\sqrt{4kTR}\sqrt{3×10²}=1.57 μV.

Thus, the overall system rms noise is reduced by about half. This corresponds to an improvement in overall signal-to-noise (S/N) ratio of about 6 dB.

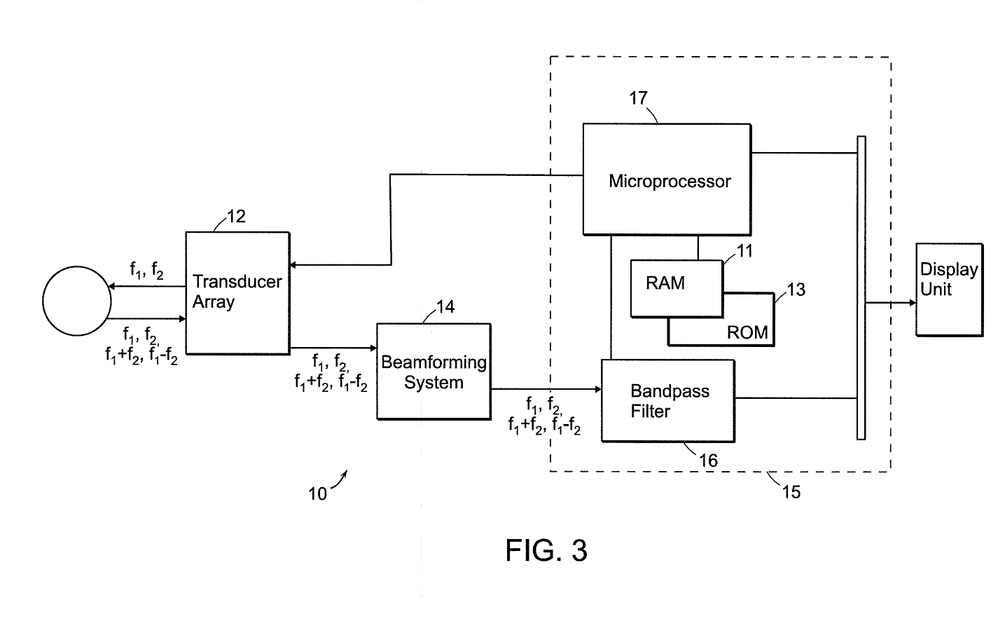

Referring to FIG. 3, an ultrasound imaging system 10 in accordance with the present invention is disclosed. The imaging system 10 includes a transducer array 12, a beamforming system 14, and a processing system 15. The transducer array 12 is electrically coupled to the beamforming system 14 and the beamforming system 14 is electrically coupled to the processing system 15.

Transducer arrays 12 and beamforming systems 14 can include those described in U.S. Pat. Nos. 6,106,472 and 6,869,401, and in U.S. application Ser. No. 10/997,062, filed Nov. 24, 2004, the above patents and application being incorporated herein by reference in their entirety. The beamforming apparatus 14 is structured and arranged to provide multiple electrical signals to the transducer array 12 to drive individual transducer elements that are disposed in the array 12. The transducer array 12 converts these electrical signals into ultrasound energy, which is transmitted into a region of interest, such as mammalian tissue. When the ultrasound energy is reflected back to the transducer array 12 from the region of interest, the transducer array 12 converts the echoed ultrasound energy back into electrical signals, which are transmitted to the beamforming system 14.

According to the present invention, the transducer array 12 transmits ultrasound signals at plural frequencies, such as at a first frequency, f₁, and at a second frequency, f₂, into the region of interest. Although FIG. 3 shows a single transducer array 12 that transmits ultrasound signals at a first frequency, f₁, and at a second frequency, f₂, plural transducers can be used.

A transducer array 12 for transmitting ultrasound signals at a first frequency, f₁, and at a second frequency, f₂, will now be described. More specifically, a 64-element, phased-array, steerable, transmit driver/applicator integrated circuit (IC) chip (hereinafter “transmit chip”) that is programmed to focus ultrasonic energy at any depth and any position is disclosed.

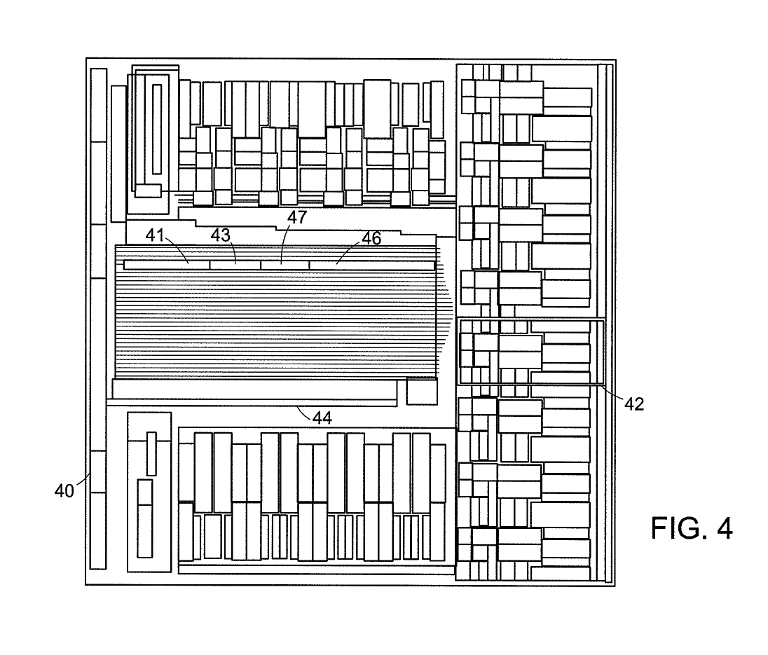

Referring to FIG. 4, there is shown a transmit chip 40 in accordance with the present invention. Mixed-mode processes are used in the transmit chip 40. More particularly, core logic, memory, timing functions, and control functions are implemented using a 5 volt(V) CMOS low-voltage (LV) process while the programmable, high-voltage driver is implemented using a 100V, CMOS high-voltage (HV) process.

The transmit chip 40 can include 16-128 channels, but in this preferred embodiment uses 64 channels. Each channel includes a level shifter that converts the low-voltage pulse sequence into a high-voltage, such as 100V, pulsed driver 42. A global, low-voltage counter 44 broadcasts master clock and bit values in each channel process. A global, low-voltage memory 46 controls transmit frequencies, pulse numbers, and pulse sequences. A plurality of transmitting integrated circuits and receiving integrated circuit each with between 16 and 128 channels per integrated circuit can be used in combination to proceed a system with from 128 to 256 channels.

Low-voltage comparators 41 provide a ten-bit delay selection for each channel. Low-voltage frequency counters 43 provides programmable transmit frequencies. Low-frequency pulse counters 47 provide different pulse sequences. These features enable programming the transmit chip 40 with more than one set, i.e., a dual set, of transmit frequencies and number of pulses.

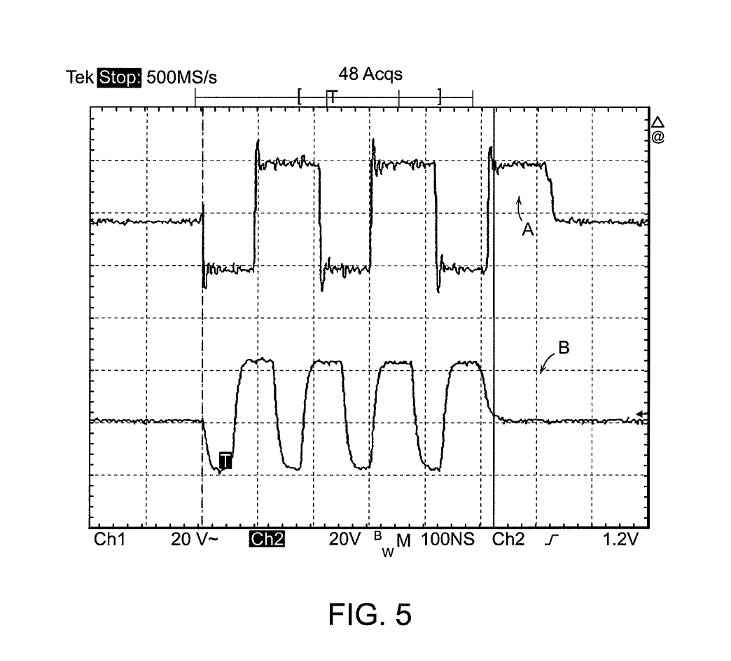

Consequently, “odd” channels of the dual sets can be structured and arranged to use an “A” set of transmit frequencies and number of pulses while “even” channels of the dual sets can be structured and arranged to use a “B” set of transmit frequencies and number of pulses. FIG. 5 shows oscilloscope traces of transmitted waveforms of two channels. The first channel, i.e., the “A” set: 3 pulses at 4.75 MHz. The second channel, i.e., the “B” set: 4 pulses at 8 MHz.

However, with this arrangement, a 128-channel transmit chip is required. Channels 1-64 receive the “A” set and channels 65-128 receive the “B” set.

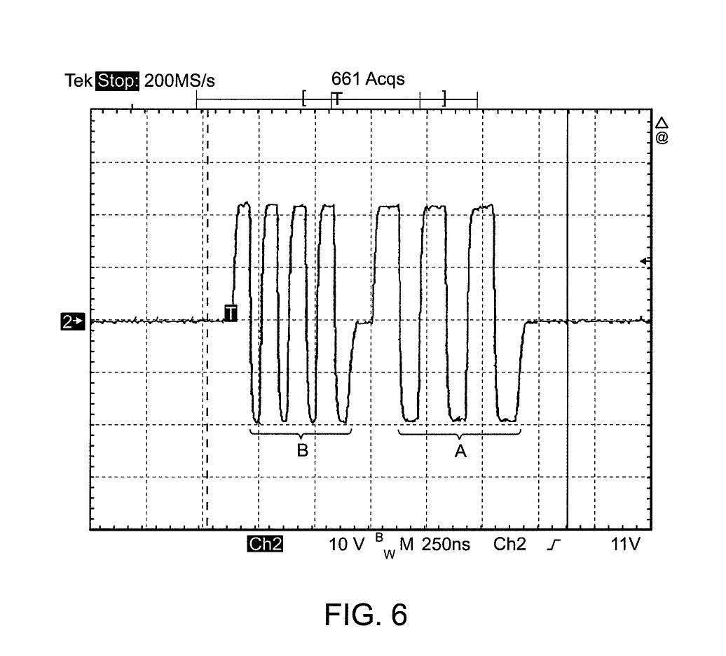

The transmit chip 40 of the present invention, however is programmable to transmit the dual, i.e., “A” and “B”, sets in a single waveform. For example, FIG. 6 shows oscilloscope traces of transmitted waveforms on a single channel with back-to-back transmit pulse trains consisting of different frequencies and different numbers of pulses for set “A” and set “B”. Accordingly, two transmit pulse trains, that can be focused at different locations in the region of interest, can be output on the same channel (or a separate channel) within the same transmission sequence.

With this arrangement, a single 64-channel transmit chip 40 is required. Each of the 64 channels receives one of the 64 dual, “A” and “B”, sets.

Alternatively, the disclosed transmit chip 40 can be operated in a mode in which the transmit waveform can be generated off-chip and, then input into the transmit chip 40 via digital programming pins.

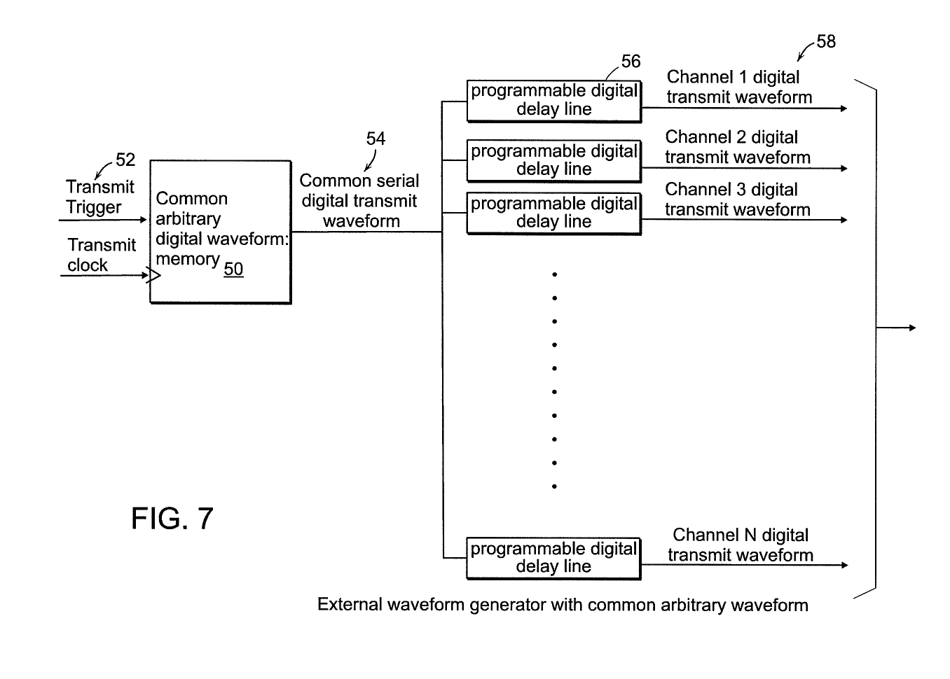

The external waveform generator can be implemented in one of two embodiments. In the first embodiment there is a common waveform memory and a multitude of programmable digital delay lines, one for each transmit channel. The digital arbitrary waveform is stored in the common memory 50 (FIG. 7) and is shifted out serially following a transmit trigger pulse 52. The serial waveform 54 is then passed through individual programmable digital delay lines 56 to achieve the intended transmit delays 58 among all channels.

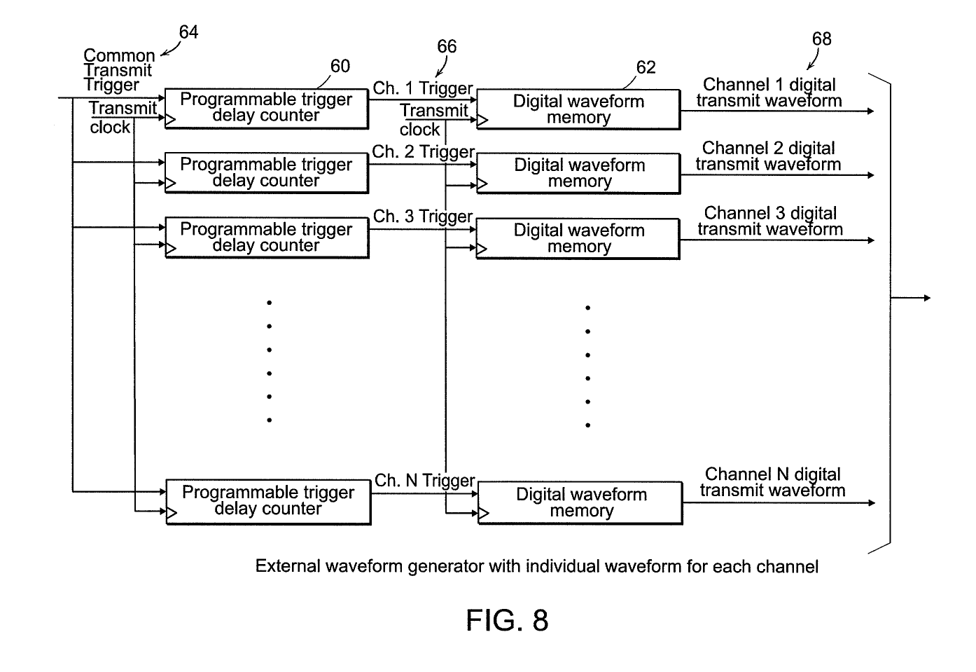

In the second embodiment, there is a multitude of sets of programmable trigger delay counters 60 and transmit waveform memories 62, one set for each channel as shown in FIG. 8. A common transmit trigger pulse 64 starts all trigger delay counters. These counters produce individual channel transmit triggers 66 after the programmed delay count for that channel is reached. Each individual channel trigger then triggers the serial read out of the transmit waveform 68 from the individual transmit waveform memory. The channel transmit waveforms are then sent to the transmitting integrated circuit 40.

The first embodiment produces a common transmit waveform for all channels, but each channel can be individually programmed for a different delay. The second embodiment also provides individual channel delay programming, and in addition, can produce a different wave shape for individual channels. Both embodiments can be configured using a FPGA (Field Programming Gate-Array), or can be implemented using an application specific integrated circuit (ASIC).

Thus, two separate 64-channel transmit chips 40 can be used to control a 128-element transducer array 12, permitting two different waveforms to be transmitted at different frequencies and having different pulse numbers to two different locations in the region of interest.

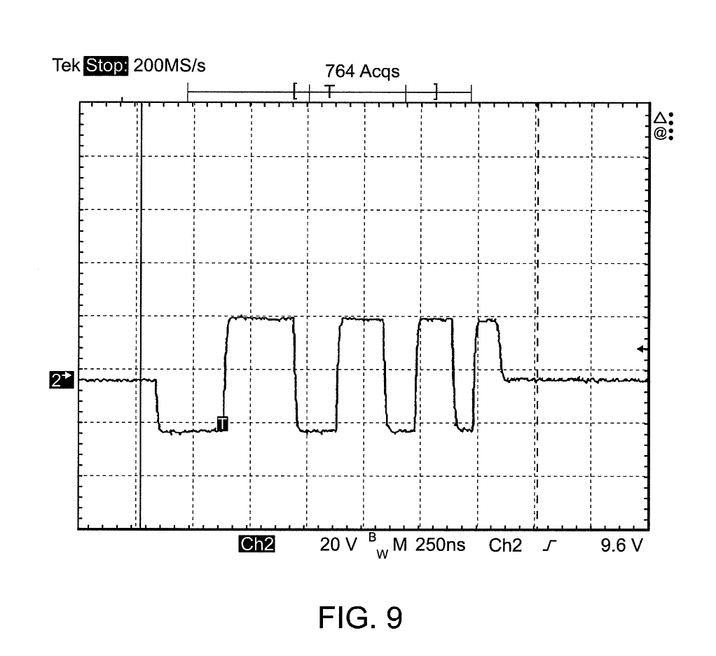

FIG. 9 shows an oscilloscope trace of an arbitrary, transmitted waveform. The disadvantages of an arbitrary system include only one channel per channel pair can be used, hence, the maximum number of pulses in this example is limited to 64; and the maximum and minimum pulse high or pulse low times are 1 G-clock and 17 G-clocks, respectively.

The beamforming system 14 provides a beamformed signal to the processing system 15. The processing system 15 processes the beamformed signal to provide ultrasound image data for display on a display unit 18. Although FIG. 3 shows the beamforming system 14 and the processing system 15 as two distinct elements, the beamforming system 14 can alternatively, be an element of the processing system 15, or vice versa, without violating the scope and spirit of this disclosure.

The processing system 15 can be structured and arranged to receive linear and non-linearly-generated (beamformed) signals that correspond to ultrasound signals corresponding to the first frequency, to the second frequency, and to a third frequency that is a function of the first frequency and the second frequency, i.e., ƒ₁, ƒ₂, ƒ₁−ƒ₂, ƒ₁+ƒ₂.

The processing system 15 includes a high-frequency bandpass filter 16, such as a high-frequency notch filter, which are well known to the art. The bandpass filter 16 receives the beamformed signals and passes those signals within a band of frequencies proximate to the bandwidth of the non-linearly-generated, mixing term, ƒ₁+ƒ₂. The bandpass filter 16 attenuates or suppresses those signals at frequencies higher than and lower than the band of frequencies passed, i.e., ƒ₁, ƒ₂, and ƒ₁−ƒ₂.

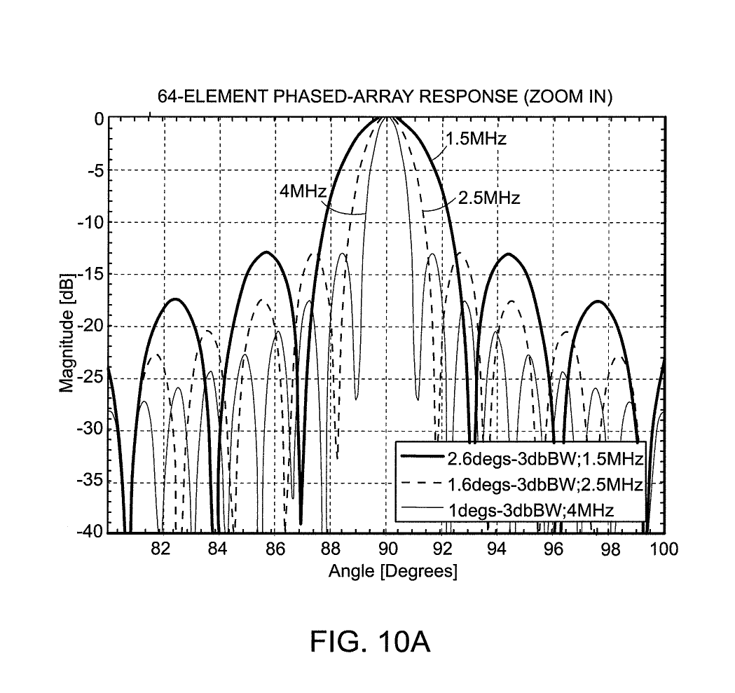

The passed frequencies, which comprise quadratic or higher order terms, have a narrower beam width than either of the individual image signal at the first frequency, f₁, or the image signal at the second frequency, f₂. FIG. 10a shows the narrowing beam width associated with higher frequencies. More particularly, FIG. 10a shows an array response, i.e., a beam pattern, at 1.5 MHz, 2.5 MHz, and 4 MHz. The main lobe of the array response at the high-frequency, mixing term, i.e., 4 MHz, than either of the original 1.5 MHz or 2.5 MHz beams. The narrower beam width of the higher-frequency mixing signal provides improved image resolution.

A preferred embodiment of a method 80 of operating an ultrasound system in accordance with the invention is illustrated in connection with FIG. 10b. The method can be performed using a software program on a computer or system processor. The user can initiate the scan 82 using a computer interface, such as, a keyboard or touchscreen. After setting the scan line number to zero 84, or other initiating line number, the scan line profile is acquired 86 and a waveform type is selected 87. This can be a standard single frequency scan 89, a dual frequency scan 90, or a hybrid scan 88 that can be selected by the user. The scan line is then transmitted 92 and the data is received 94. The program then determines whether additional lines need to be acquired 95 and increments the scan line 96, or alternatively indicates that the frame is complete 98.

The processing system 15 further includes a processing sequence stored on a computer readable medium, memory 11 and 13, and a processing unit 17. The processing unit 17 controls operation of the processing system 15, the beamforming system 14, and the transducer array 12. More specifically, the processing unit controls the transfer of ultrasound image data from the transducer arrays 12 to the display unit 18. Control is based on user input originating from an input/output device and/or on the processing sequence, i.e., driver program or application.

Memory in the processing system 15 can include non-volatile memory, such as read-only memory (ROM) 13, and volatile memory, such as random access memory (RAM) 11. ROM 13 can include, without limitation, stored data, such as look-up tables and the like, and applications and/or driver programs, i.e., the processing sequences, that are executable by the processing unit 17. RAM 11 includes storage space on which the processing unit 17 can store imaging or other data temporarily and can execute applications and/or driver programs.

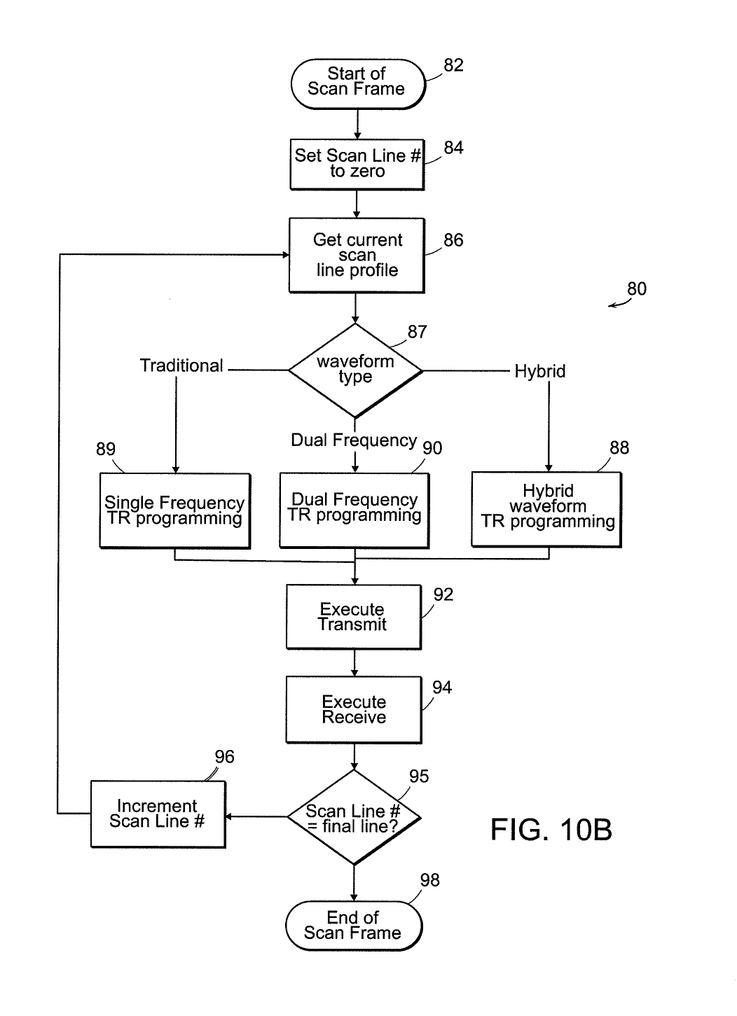

A preferred embodiment of an ultrasound imaging system 100 in accordance with the invention is illustrated in FIG. 11. FIG. 11 is a schematic functional block of one embodiment of the ultrasound imaging system similar to systems are described in U.S. Pat. No. 5,957,846 to Alice M. Chiang et al., issued Sep. 28, 1999, entitled “Portable Ultrasound Imaging System,” the entire contents of which is incorporated herein by reference. As shown, the system 100 includes an ultrasonic transducer array 140 which transmits ultrasonic signals into a region of interest or image target 120, such as a region of human tissue, and receives reflected ultrasonic signals returning from the image target. The system 100 also includes a front-end interface or processing unit 180 which is connected by cable 160, for example, a coaxial cable to the transducer array 140 and includes a transducer transmit/receive control chip 220.

Ultrasonic echoes reflected by the image target 120 are detected by the ultrasonic transducers in the array 140. Each transducer converts the received ultrasonic signal into a representative electrical signal which is forwarded to an integrated chip having preamplification circuits and time-varying gain control (TGC) circuitry 300. The preamplification circuitry sets the level of the electrical signals from the transducer array 140 at a level suitable for subsequent processing, and the TGC circuitry is used to compensate for attenuation of the sound pulse as it penetrates through human tissue and also drives the beamforming circuits 320 to produce a line image. The conditioned electrical signals are forwarded to the beamforming circuitry 320 which introduces appropriate differential delays into each of the received signals to dynamically focus the signals such that an accurate image can be created. Further details of the beamforming circuitry 320 and the delay circuits used to introduce differential delay into received signals and the pulses generated by a pulse synchronizer are described in U.S. Pat. No. 6,111,816 to Alice M. Chiang et al., issued Aug. 29, 2000 entitled “Multi-Dimensional Beamforming Device,” the entire contents of which is incorporated herein by reference.

A memory 300 stores data from a controller 280. The memory 300 provides stored data to the transmit/receive chip 220, the TGC 30 and the beamformer 320. The output from the system controller 280 is connected directly to a custom or Fire Wire Chipset. The FireWire Chip set is described in U.S. Pat. No. 6,869,401 entitled “Ultrasound Probe with Integrated Electronics,” by Jeffrey M. Gilbert et al., the entire contents of which is incorporated herein by reference. “FireWire” refers to IEEE standard 1394, which provides high-speed data transmission over a serial link. There also exists a wireless version of the FireWire standard allowing communication via a wireless link for untethered operation.

The FireWire standard is used for multimedia equipment and allows 100-200 Mbps and preferably in the range of 400-800 Mbps operation over an inexpensive 6 wire cable. Power is also provided on two of the six wires so that the FireWire cable is the only necessary electrical connection to the probe head. A power source such as a battery or IEEE 1394 hub can be used. The FireWire protocol provides both isochronous communication for transferring high-rate, low-latency video data as well as asynchronous, reliable communication that can be used for configuration and control of the peripherals as well as obtaining status information from them. Several chipsets are available to interface custom systems to the Fire Wire bus. Additionally, PCI-to-FireWire chip sets and boards are currently available to complete the other end of the head-to-host connection. CardBus-to-Firewire boards can also be used.

Although the VRAM controller directly controls the ultrasound scan head, higher level control, initialization, and data processing and display comes from a general purpose host such as a desktop PC, laptop, or palmtop computer. The display can include a touchscreen capability. The host writes the VRAM data via the VRAM Controller. This is performed both at Initialization as well as whenever any parameters change (such as number or positions of zones, or types of scan head) requiring a different scanning pattern. During routine operation when data is just being continually read from the scan head with the same scanning parameters, the host need not write to the VRAM. Because the VRAM controller also tracks where in the scan pattern it is, it can perform the packetization to mark frame boundaries in the data that goes back to the host. The control of additional functions such as power-down modes and querying of buttons or dial on the head can also be performed via the Fire Wire connection.

Although FireWire chipsets manage electrical and low-level protocol interface to the Fire Wire interface, the system controller has to manage the interface to the FireWire chip set as well as handling higher level Fire Wire protocol issues such as decoding asynchronous packets and keeping frames from spanning isochronous packet boundaries.

Asynchronous data transfer occurs at anytime and is asynchronous with respect to the image data. Asynchronous data transfers take the form of a write or read request from one node to another. The writes and the reads are to a specific range of locations in the target node's address space. The address space can be 48 bits. The individual asynchronous packet lengths are limited to 1024 bytes for 200 Mbps operation. Both reads and writes are supported by the system controller. Asynchronous writes are used to allow the host to modify the VRAM data as well as a control word in the controller which can alter the operation mode. Asynchronous reads are used to query a configuration ROM (in the system controller FPGA) and can also be used to query external registers or I/O such as a “pause” button. The configuration ROMs contain a querible “unique ID” which can be used to differentiate the probe heads as well as allow node-lockings of certain software features based on a key.

Using isochronous transfers, a node reserves a specified amount of bandwidth and it gets guaranteed low-overhead bursts of link access every 1/8000 second. All image data from the head to the host is sent via isochronous packets. The FireWire protocol allows for some packet-level synchronization and additional synchronization is built into the system controller.

The front-end processing or interface unit system controller 280 interfaces with a host computer 200, such as a desktop PC, laptop or palmtop, via the custom or FireWire Chipsets 240, 340. Note that other standard communication interfaces, such as, a universal serial bus (USB) can also be used. This interface allows the host to write control data into the memory 260 and receive data back. This may be performed at initialization and whenever a change in parameters such as, for example, number and/or position of zones, is required when the user selects a different scanning pattern. The front-end system controller 280 also provides buffering and flow control functions, as data from the beamformer is sent to the host via a bandwidth-constrained link, to prevent data loss.

The host computer 200 includes a keyboard/mouse controller 380, and a display controller 420 which interfaces with a display or recording device 440. A graphical user interface described in U.S. Pat. No. 6,669,633 entitled “Unitary Operator Control for Ultrasonic Imaging Graphical User Interface,” by Michael Brodsky, the entire contents of which is incorporated herein by reference, may be used in a preferred embodiment of the present invention.

The host computer further includes a processing unit such as microprocessor 360. In a preferred embodiment of the ultrasound imaging system in accordance with the present invention the microprocessor 360 includes on-chip parallel processing elements. In a preferred embodiment, the parallel processing elements may include a multiplier and an adder. In another preferred embodiment, the processing elements may include computing components, memories, logic and control circuits. Depending on the complexity of the design, the parallel processing elements can execute either SIMD or Multiple Instruction Multiple Data (MIMD) instructions.

Further, the host computer includes a memory unit 400 that is connected to the microprocessor 360 and has a sequence of instructions stored therein to cause the microprocessor 360 to provide the functions of down conversion, scan conversion, M-mode, and Doppler processing which includes color flow imaging, power Doppler and spectral Doppler, and any post-signal processing. The down conversion or mixing of sampled analog data may be accomplished by first multiplying the sampled data by a complex value and then filtering the data to reject images that have been mixed to nearby frequencies. The outputs of this down-conversion processing are available for subsequent display or Doppler processing.

The scan conversion function converts the digitized signal data from the beamforming circuitry 320 from polar coordinates (r,8) to rectangular coordinates (x,y). After the conversion, the rectangular coordinate data can be forwarded for optional post signal processing where it is formatted for display on the display 440 or for compression in a video compression circuit. Scan conversion and beamforming and associated interfaces are described in U.S. Pat. No. 6,248,073 to Jeffrey M. Gilbert et al., issued on Jun. 19, 2001, entitled “Ultrasound Scan Conversion with Spatial Dithering,” the entire contents of which are being incorporated herein by reference.

The Doppler processing (CFI, PD, spectral Doppler) is used to image target tissue 120 such as flowing blood. In a preferred embodiment, with pulsed Doppler processing, a color flow map is generated. In a preferred embodiment, the CF1, PD, Spectral Doppler computation can be carried out in software running on the host processor. Parallel computation units such as those in the Intel® Pentium® and Pentium® III's MMX™ coprocessors allow rapid computation of the required functions. For parallel processing computation, a plurality of microprocessors are linked together and are able to work on different parts of a computation simultaneously. In another preferred embodiment, digital Signal Processor (DSP) can also be used to perform the task. Such arrangement permits flexibility in changing digital signal processing algorithms and transmitting signals to achieve the best performance as region of interest is changed.

Single Instruction Multiple Data (SIMD) parallel processors allow one microinstruction to operate at the same time on multiple data items to accelerate software processing and thus performance. One chip provides central coordination in the SIMD parallel processing computer. Currently, SIMD allows the packing of four single precision 32-bit floating point values into a 128-bit register. These new data registers enable the processing of data elements in parallel. Because each register can hold more than one data element, the processor can process more than one data element simultaneously. In a preferred embodiment of the present invention, all the data is organized efficiently to use SIMD operations. In a particular embodiment, Multiple Instruction Multiple Data (MIMD) parallel processors may be used, which include a plurality of processors. Each processor can run different parts of the same executable instruction set and execute these instructions on different data. This particular embodiment employing MIMD may be more flexible than the embodiment utilizing SIMD, however may be more expensive. All the kernel functions such as demodulation, Gauss match filtering, Butterworth high pass filtering, auto-correlation calculation, phase-shift calculation, frame averaging, color-averaging, spatial domain low-pass filtering, and scan conversion interpolation are implemented with SIMD or MIMD. The Doppler processing results in the processed data being scan converted wherein the polar coordinates of the data are translated to rectangular coordinates suitable for display or video compression.

The control circuit, preferably in the form of a microprocessor 360 inside of a personal computer (e.g., desktop, laptop, palmtop), controls the high-level operation of the ultrasound imaging system 100. The microprocessor 360 or a DSP initializes delay and scan conversion memory. The control circuit 360 controls the differential delays introduced in the beamforming circuitry 320 via the memory 260.

The microprocessor 360 also controls the memory 400 which stores data. It is understood that the memory 400 can be a single memory or can be multiple memory circuits. The microprocessor 360 also interfaces with the post signal processing functional instructions and the display controller 440 to control their individual functions. The display controller 440 may compress data to permit transmission of the image data to remote stations for display and analysis via a transmission channel. The transmission channel can be a modem or wireless cellular communication channel or other known communication method.

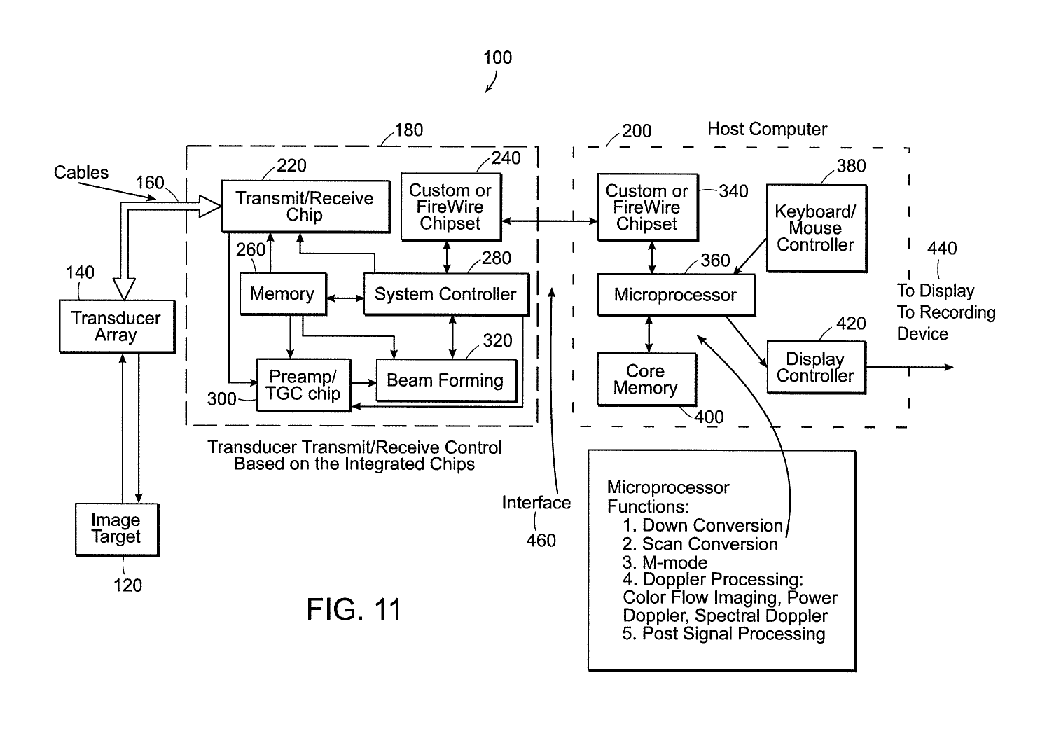

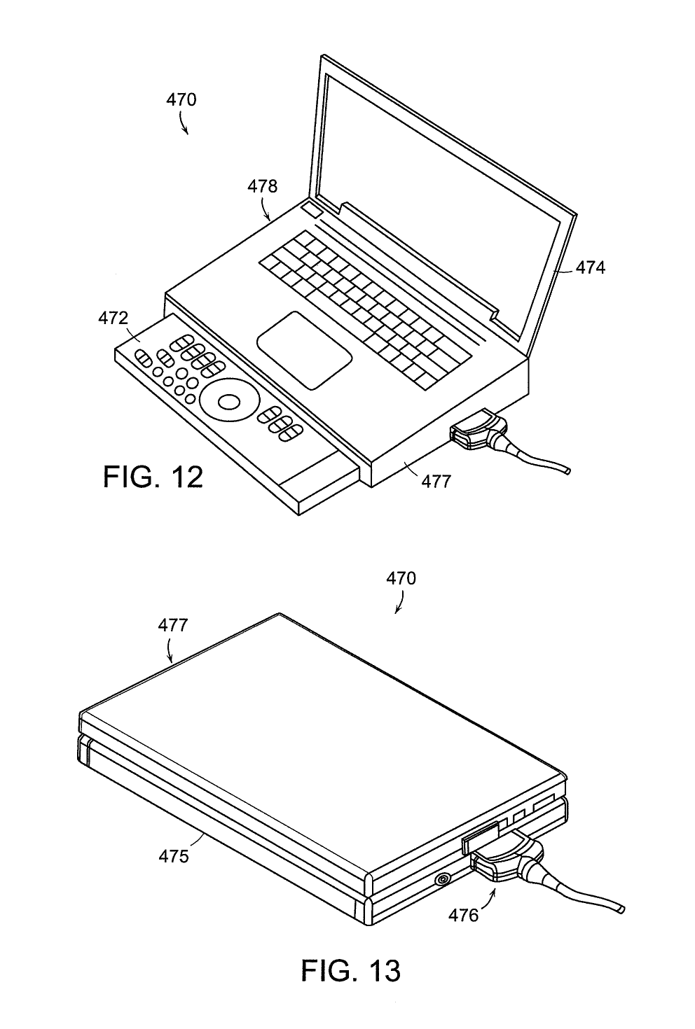

Another preferred embodiment of a system 470 is illustrated in FIG. 12 where an ultrasound operator console 472 and hot-swappable transducer ultrasound engine, such as interface 180, are integrated in a housing 477 with an off-the-shelf notebook computer 474 in a single integrated package, without hard modification to the notebook computer. The ultrasound engine and operator console are in housing 477 with similar dimensions to the notebook computer. FIG. 12 shows the system with the console extended. FIG. 13 illustrates the system 470 in the storage position with the display folded 477 and the ultrasound user interface or console in the storage position 475. An ultrasound transducer cable 476 is connected at a connector 479 in the console housing 472.

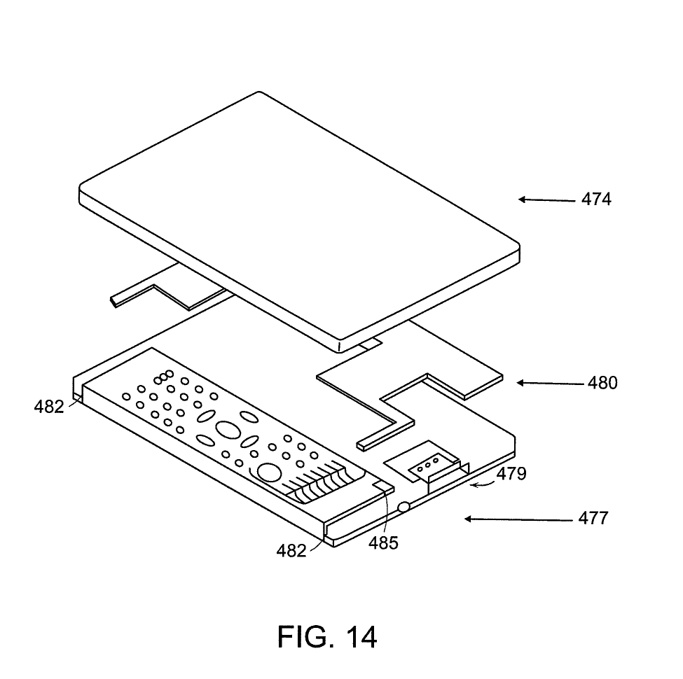

As seen in FIG. 14, the computer is attached to a mounting plate or fixture 480 which can be attached to the bottom of computer 474 with a removable adhesive. The housing 477 can then be mounted to the plate 480 with a fastener such as screws. The mounting plate provides mechanical mounting screw pems to which the engine/console package is attached using the screws. This method of attaching the engine/console to the notebook computer is completely reversible so that the notebook computer can be reverted to its original configuration. This allows an easy swapping of computer for upgrade or for repair. The operator console is mounted on sliding rails 482 in the engine/console housing 477. When in use, the operator console slides out to expose the control surface. The operator console 472 typically has a mix of button switches, rotary knobs, slide potentiometers, and trackball.

The engine/console package is electrically attached to the notebook computer via standard FireWire and or USB cables, and once again does not require any hard modifications to the notebook computer.

A sensor 485 is integrated to the operator console to detect whether the console is in the stowed or storage position or in the extended or operating position. The position information is checked by the ultrasound software to switch between two sets of software GUI. When the console is stowed, all operator controls are performed via the notebook computer keyboard 478 and touch pad. When the console is extended, the ultrasound software automatically changes the software GUI to use the controls on the console 472.

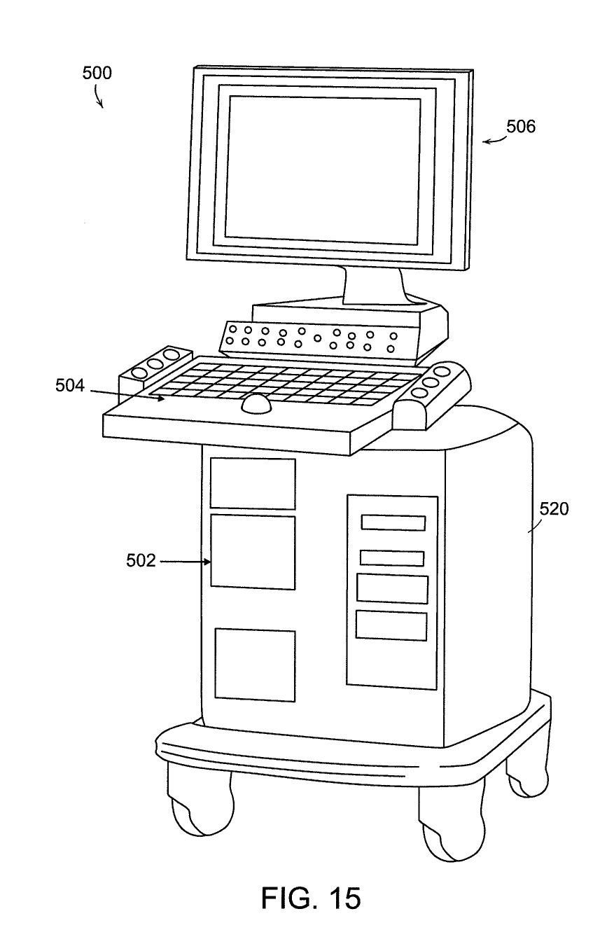

Another preferred embodiment of the invention is illustrated in the cart system 500 of FIG. 15. The system 500 has an integrated interface 180 and personal computer 502 mounted in the cart housing 520 which can be mounted on wheels. The display 506 is mounted above the ultrasound keyboard console 504.

Many changes in the details, materials, and arrangement of parts, herein described and illustrated, can be made by those skilled in the art in light of teachings contained hereinabove. Accordingly, it will be understood that the following claims are not to be limited to the embodiments disclosed herein and the equivalents thereof, and can include practices other than those specifically described.

1. An ultrasound imaging system comprising:
a transducer array that provides imaging signals at a first frequency, f₁, and at a second frequency, f₂, into a region of interest;
a beamforming system that processes image data at a third frequency that is a function of the first frequency, f₁, and second frequency, f₂; and
a processing system, including a processing sequence stored on a computer readable medium, for signal processing of an ultrasound image, the processing sequence using the function of the first frequency, f₁, and second frequency, f₂.

2. The ultrasound imaging system of claim 1, wherein the function of the first frequency, f₁, and second frequency, f₂, is a non-linearly-generated, mixing term, f₁+f₂.

3. The ultrasound imaging system of claim 1, wherein the third frequency has a narrower beam width than beam widths associated with either of the imaging signal at a first frequency, f₁, or the imaging signal at a second frequency, f₂.

4. The ultrasound imaging system of claim 2, wherein the non-linearly-generated, mixing term, f₁+f₂, is a quadratic or higher order term.

5. The ultrasound imaging system of claim 1, wherein the processing system includes a bandpass filter that passes a band of frequencies proximate to a bandwidth of the third frequency and attenuates frequencies higher and lower than the band of frequencies passed.

6. The system of claim 1 wherein the system comprises a portable ultrasound system in which the transducer array, the beamforming system and the processing system has a weight of 15 lbs. or less.

7. The ultrasound imaging system of claim 1 wherein the beamforming system and the processing system are mounted on a mobile cart.

8. The ultrasound imaging system of claim 1 wherein the processing system stores a processing sequence in a memory, the memory being connected to a processor that processes ultrasound image data with the processing sequence.

9. The ultrasound imaging system of claim 8 further comprising a scan conversion processing sequences.

10. The ultrasound imaging system of claim 8 further comprising a Doppler processing sequence.

11. The ultrasound imaging system of claim 1 wherein the processing system further comprising a processing sequence that processes with a third frequency that is a non-linearly-generated, higher-order, mixing term, f₁+f₂.

12. The ultrasound imaging system of claim 11 further comprising a bandpass filter having a pass band that passes a selected frequency that corresponds to the function of the first frequency and the second frequency.

13. The ultrasound imaging system of claim 11 further comprising an interface that houses the beamforming system and that has a connector that connects to a cable to the transducer array.

14. The ultrasound imaging system of claim 1 wherein the processing system includes a bandpass filter that passes a band of frequencies proximate to a bandwidth of the non-linearly-generated, higher-order, mixing term, f₁+f₂, and attenuates frequencies higher and lower than the band of frequencies passed.

15. An ultrasound imaging system comprising:
a transducer array that provides imaging signals at a first frequency, f₁, and at a second frequency, f₂, into a region of interest;
a beamforming system that processes image data at a third frequency that is a function of the first frequency, f₁, and second frequency, f₂; and
a bandpass filter that passes a band of frequencies proximate to the bandwidth of the third frequency and attenuates frequencies higher and lower than the band of frequencies passed.

16. The ultrasound imaging system of claim 15 wherein the function of the first frequency, f₁, and second frequency, f₂ is a non-linearly-generated, mixing term, f₁+f₂.

17. The ultrasound imaging system of claim 16 wherein the non-linearly-generated, mixing term, f₁+f₂, is a quadratic or higher order term.

18. The ultrasound imaging system of claim 15 wherein the third frequency has a narrower beam width than beam widths associated with either of the imaging signal at the first frequency, f₁, or the imaging signal at the second frequency, f₂.

19. The ultrasound imaging system of claim 15 further comprising a processing system having a processing sequence stored on a computer readable medium for signal processing of an ultrasound image.

20. The ultrasound imaging system of claim 15, wherein the third frequency has a narrower beam width than beam widths associated with either of the imaging signal at the first frequency, f₁, or the imaging signal at the second frequency, f₂.

21. The ultrasound imaging system of claim 15, wherein the non-linearly-generated, higher-order, mixing term, f₁+f₂, is a quadratic term.

22. The ultrasound imaging system of claim 15, further comprising a processing system having a processing sequence stored on a computer readable medium for signal processing of an ultrasound image.

23. A bandpass filter for an ultrasound imaging system, the ultrasound imaging system having a transducer array that provides imaging signals at a first frequency, f₁, and at a second frequency, f₂, into a region of interest; and a beamforming system for processing image data at a third frequency, having a bandwidth, that is a function of the first frequency, f₁, and second frequency, f₂,
the bandpass filter having a pass band of frequencies proximate to the bandwidth of the third frequency and attenuates frequencies higher and lower than the band of frequencies passed.

24. The bandpass filter of claim 23 wherein the function of the first frequency, f₁, and second frequency, f₂, is a non-linearly-generated, higher-order, mixing term, f₁+f₂.

25. The bandpass filter of claim 24 wherein the non-linearly-generated, higher-order, mixing term, f₁+f₂, is a quadratic term.

26. A method of ultrasound imaging comprising:
delivering signals at a first frequency, f₁, and at a second frequency, f₂, into a region of interest;
receiving an echo signal from the region of interest; and
processing the echo signal using image data at a third frequency that is a function the first frequency, f₁, and the second frequency, f₂.

27. The method of claim 26, wherein the processing step includes processing the echo signal using image data at a third frequency that is a non-linearly-generated, mixing term, f₁+f₂.

28. The method of claim 27, wherein the processing step includes processing the echo signal using image data at a third frequency that is a non-linearly-generated, higher order, mixing term, f₁+f₂.

29. The method of claim 28, wherein the processing step includes processing the echo signal using image data at a third frequency that is a non-linearly-generated, quadratic, mixing term, f₁+f₂.

30. The method of claim 26, wherein the processing step includes filtering the echo signal to pass a band of frequencies proximate to the bandwidth of the image data at the third frequency and to attenuate frequencies higher and lower than the band of frequencies passed.

31. The method of claim 26 further comprising:
receiving an echo signal from a region of interest resulting from imaging signal transmitted at a first frequency, f₁, and at a second frequency, f₂, into a region of interest; and
processing the echo signal at a third frequency that is a function of the first frequency, f₁, and the a second frequency, f₂.

32. The method of claim 31, wherein the processing step includes performing scan conversion.

33. The method of claim 32, wherein the processing step performing Doppler processing.

34. The method of claim 33, wherein the processing step includes filtering with a bandpass filter.

35. The method of claim 34, wherein the processing step includes filtering the echo signal to pass a band of frequencies proximate to the bandwidth of the third frequency and to attenuate frequencies higher and lower than the band of frequencies passed.

36. The system of claim 1, wherein the beamforming system is in an interface connected to the processing system using a standard communication connection, such as, Firewire or USB.

37. The system of claim 1, wherein the beamforming system and processing system are mounted in a cart.

38. The system of claim 1, wherein the system has a weight of less than 15 pounds.

39. The system of claim 1 further comprising a moveable ultrasound console in a housing attached to the processing system.

40. The system of claim 1, wherein the processing system comprises a laptop computer attached to the housing.
